(19) United States
(12) Patent Application Publication
(10) Pub. No.: US 2018/0028810 A1
(43) Pub. Date: Feb. 1, 2018

(54) SYSTEM FOR CONTROLLING STIMULATION IMPULSES

(71) Applicant: WEARABLE LIFE SCIENCE GMBH, Nümberg (DE)

(72) Inventors: Philipp G. SCHWARZ, Frankfurt (DE); Nordin KOUACHE, Frankfurt (DE); Shahid MEHBOOB, Frankfurt (DE)

(21) Appl. No.: 15/551,980

(22) PCT Filed: Feb. 18, 2016
(86) PCT No.: PCT/EP2016/053489

§ 371 Date: Aug. 18, 2017

(30) Foreign Application Priority Data
Feb. 18, 2015 (DE) 20 2015 001 313.9
Feb. 27, 2015 (DE) 10 2015 002 484.1
Feb. 27, 2015 (DE) 10 2015 002 565.1
Aug. 14, 2015 (DE) 20 2015 005 645.8
Feb. 12, 2016 (EP) PCT/EP2016/000236

(51) Int. Cl. A61N 1/36; A61B 5/145; A41D 1/00; A61B 5/00; A61N 1/04; A61B 5/053; A61B 5/0488

(52) U.S. Cl. CPC A61N 1/36031; A61B 5/053; A61B 5/14542; A61B 5/0488; A61B 5/4866; A61B 5/4836; A61B 5/6804; A61N 1/0484; A61N 1/0476; A61N 1/0452; A61N 1/36003; A41D 1/002; A61B 2505/09; G06F 19/345

(57) ABSTRACT

A system for controlling stimulation impulses, including at least one control unit and one item of clothing having a plurality of electrodes for electro-stimulation. The control unit is configured to carry out electro-stimulation with defined parameters at different electrodes and, during a training session, different parameters can be produced at different electrodes by said control unit.

### FIELD OF THE INVENTION

The present invention relates to a system for controlling stimulation impulses.

### BACKGROUND OF THE INVENTION

From prior art stimulation impulses are known, in particular an electrical muscle stimulation (EMS) for the stimulation of different biological tissues such as muscles and nerves.

In the case of electrical muscle stimulation often an item of clothing is used in which the required electrodes are integrated in a detachable or permanent manner. In the case of high-quality modern EMS systems a high number of electrodes is used. In doing so, accordingly, the expenditure with respect to the electric and/or electronic control unit increases. Also for the transmission of the impulses of the electrostimulation to the control unit a high number of electrical lines is required. Since also an appreciable power has to be transmitted with it, a correspondingly large cross-section of the line is necessary. Accordingly, a high expenditure for the integration of the lines into the clothing is required.

### SUMMARY OF THE INVENTION

It is the object of the present invention to provide a system for electrostimulation which can drive different electrodes in a simplified manner and can carry out different electrostimulations, in particular dependent on the position of the respective electrode.

This object is solved by the features of patent claim 1. Preferable embodiments are the subject matter of the dependent claims.

A system for controlling stimulation impulses comprises at least one control unit and at least one item of clothing comprising a plurality of electrodes for electrostimulation. The control unit is configured to carry out electrostimulations with defined parameters at different electrodes, and, during a training session, different parameters can be produced at different electrodes by means of said control unit. The training session may comprise a cycle of several minutes, wherein phases of stimulation of, for example, 3 seconds alternate with rest periods of the same duration. Said control unit is configured to effect different impulses at several electrodes. In this sense, the control unit preferably comprises a data processing unit which is configured to specifically define for different electrodes at one time point parameters for the electrostimulation. A module for generating the electrostimulation generates dependently on these ideal values the desired signal of the EMS. A switch unit may be provided for connecting the desired electrode(s) being fixed at the body of the exercising person with the signal of the electrostimulation. The switching by the switch unit and the generation of the signals of the electrostimulation are carried out in a coordinated manner so that therewith with a single unit for generating the electrostimulation profiles of individual electrostimulations can be transmitted to different electrodes.

In particular, the control unit comprises at least one generating device for generating impulses of the electrostimulation, and a switch device is configured to switch the electrostimulation over to the desired electrodes and/or to distribute it to the desired different electrodes.

The parameters of the electrostimulation are the impulse type, the frequency, the intensity, the polarity, the duration of the impulse and the rest period between impulses. And in one embodiment during a training session, in particular, only the parameters impulse type, frequency, the intensity and/or the polarity can be changed. The last-mentioned three parameters are directly connected with the generation of the electrostimulation. On the contrary, the parameters duration of the impulse and rest period between the impulses are determined or can be determined by the switch unit (and/or distributing device) which switches over between the different electrodes.

A system for controlling stimulation impulses during a stimulation at a user may comprise at least one sensor, at least one data processing unit and at least one impulse unit. In this case the at least one sensor is suitable for measuring at least one measuring value. The data processing unit is configured to compare the measuring values with one threshold value each and to generate control signals for the impulse unit, when the measuring value(s) and the threshold values are in a predefinable ratio to each other. The impulse unit is suitable for triggering stimulation impulses and it is configured to change one or more stimulation impulse parameters dependently on the control signal. The combination of a stimulation impulse with the stimulation impulse parameters can also be referred to as an electrostimulation.

In a respective system the control unit in particularly comprises a first mode which can in particularly be referred to as a learner mode and, in addition, a second mode which can particularly be referred to as an expert or trainer mode. For at least one parameter of the electrostimulation the adjustable range of values in the first mode is smaller than in the second mode. Through this a non-experienced user is protected from setting exercise parameters at said system which are not suitable, individually or generally, for him or her. For example, in the case of the back musculature for strengthening the deep musculature a high frequency should be used. At this location a frequency which is too low may even be suboptimal with respect to muscle growth. In the expert mode such restrictions are omitted.

In addition, the system may comprise at least one sensor and the control unit may be configured to change at least one parameter of the electrostimulation dependently on measuring results of this sensor. Here, there is a plurality of possible ways of exerting influence. For example, with the help of a pulse sensor or a respiratory frequency sensor the state of exhaustion of the exercising person can be identified and correspondingly the stimulation can be reduced. Also via e.g. a conductivity sensor (contact resistance) the transfer from the respective electrode to the skin can be examined and, dependently on the result, the electrostimulation can be adjusted.

In particular, one electrode or a plurality of electrodes can non-interchangeably be assigned to one or more channels. For this electrode or these electrodes at least one parameter of the electrostimulation can be envisaged in one mode of operation, in particular a learner mode, as opposed to another mode, from a limited range of possibly selectable parameters. Here, the target is the feature of non-interchangeability. In this sense it has to be guaranteed that electrodes which should not be used at a special location of the body can only be connected with the system in such a manner that a mix-up can be excluded. This, for example, can be achieved with the help of special electrical connectors.

In particular, the item of clothing comprises data lines for transmitting measuring values and/or control signals and further power lines for transmitting power of stimulation impulses. Here, the power lines have a larger cross-section than the data lines. In this way the total number of lines and the cabling effort can considerably be reduced. Conventionally, it is common to lead single wirings of all electrodes to one (central) control unit. By the data lines which work like a (data) bus a uniform power supply can be provided and at locations near the respective electrodes a switch which is activated by the control signals being transmitted by the data lines can provide the respective electrode with the stimulation impulse.

Preferably, the item of clothing comprises at at least two locations near one (or more) respective electrode(s) switch assemblies, wherein each switch assembly comprises at least one power switch element, such as in particular a transistor or the like, and wherein the switch assembly is configured to actuate the power switch element dependently on measuring data provided by a sensor or control information provided by the control unit, so as to provide the respective electrode(s) with an electrostimulation. Here, the switch assembly or the switch assemblies may comprise at least the sensor. In particular, the switch assemblies are fixed on the item of clothing apart of each other.

Preferably, the switch assembly may be smaller than 2 cm³ and in a further preferred embodiment smaller than 0.6 cm³. The power element may be configured as a simple switch for switching or breaking off a stimulation impulse being generated at a remote location, or it may be connected with a voltage/current supply for generating a stimulation impulse by itself.

An item of clothing may further comprise an electrode array of single electrodes, wherein the electrode array particularly comprises at least eight electrodes and the system is configured to provide, during a training session, stimulation impulses for each of these electrodes, comprising parameters which are different in groups or entirely individually. So a targeted stimulation can be achieved.

In particular, in the data processing unit a ratio for the adjustment of at least two stimulation impulse parameters may be specified. And in the case of a change of measuring values of one or more sensors the adjustment of these parameters according to this ratio may be carried out, wherein the stimulation parameters may be parameters for the same or different electrodes. This should be explained in the following example: For example, there is a ratio of 2:1 between the impulse level (e.g. voltage) of the biceps muscles and the upper leg muscles so that the upper leg muscles are activated stronger. When, for example, a measuring value which represents the activity of the exercising person, such as e.g. pulse, respiratory frequency or blood sugar value, decreases, so the activity of these muscle groups can be adjusted in the given ratio. It is possible to permanently specify different ratios for different pairs of parameters each, or it may be possible that these ratios can be adjusted by the user and/or a trainer.

In addition, more sensors can be configured to receive different measuring values. In such a case the sensors may use different measuring principles. The control unit and/or the system are configured to evaluate these measuring values by the help of comparison and to trigger stimulation impulses from this and, in doing so, to change stimulation impulse parameters. So, for example, a sensor, such as a camera, can recognize the movement of the person and, in addition, the pulse of the person is measured. Thus, when different sensors receive measuring values each which in a combined evaluation suggest that an adjustment of the electrostimulation has to be carried out, then this is carried out accordingly.

In the training wear electric lines for the power transmission are provided. In a nearer embodiment, in addition, lines for control signals are provided. With this combination a bus is obtained. Either for each EMS element individually directly at the position of the stimulator the impulse can be switched/controlled/regulated, or a plurality of small switch elements is provided with which for very small areas (similarly to a display of a screen) individually the impulses can be switched. Especially the latter case is advantageous, because so the single power switch elements can be designed very small and so they only little protrude. It is possible to measure the transition resistance to the skin for each electrode, and individually for this electrode or these electrodes the EMS power can be adjusted.

In the system one or more sensors can be configured to diagnose tensions in a muscular tissue.

The measuring principle used may be the principle of a bioelectric impedance analysis (BIA), the oxygen saturation, the electromyography and/or the calorie consumption. Then, the control unit is configured to define muscles dependently on this measuring result, which have to be activated for reducing the tensions, and the control unit may further be configured to send respective commands of the muscle electrostimulation to the electrodes which are assigned to the muscles to be activated. When, for example, it is recognized that there is a tension in the right shoulder, then, dependently on the biomedical finding, for example muscles in the right shoulder can be activated. These tensions can be reduced via a mutual compensation. Also a thermal activation may be carried out. In this case, thermal elements heating the area of the tension (in this example the right shoulder) in a targeted manner are integrated in the clothing.

### BRIEF DESCRIPTION OF THE DRAWINGS

In the following, embodiment examples of the present invention are explained in more detail with respect to the attached figures and examples. Shown are in:

FIG. 1 a schematic illustration of a portable system for controlling EMS impulses during an EMS use at an EMS user,

FIG. 2 a schematic illustration of a system for controlling stimulation impulses comprising at least two electrodes, one line for electric connection of impulse unit and electrode,

FIG. 3 a schematic illustration of an EMS user during the execution of a sequence of movements being acquired by means of a sensor and visualized on a monitor as a virtual reality application,

FIG. 4 a schematic illustration of an EMS user who is equipped with at least two electrodes,

FIG. 5 an illustration of a voltage characteristic of a stimulation impulse,

FIG. 6-9 schematic arrangements of the electrodes and sensors with respect to the control unit of the EMS system, and

FIG. 10 an illustration comprising a plurality of individually activatable sensors/electrodes.

### DETAILED DESCRIPTION OF THE INVENTION

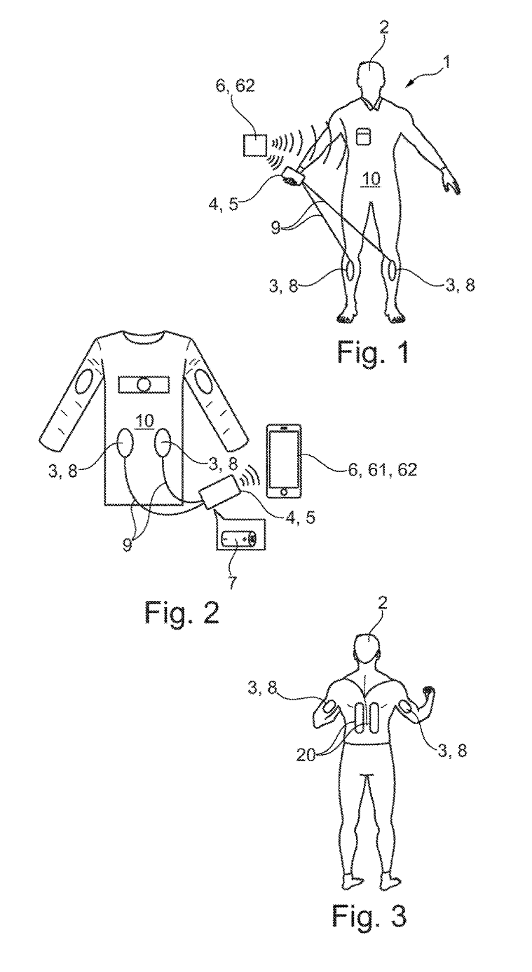

In FIG. 1 a schematic illustration of a unit/system for controlling stimulation impulses is shown. The system 1 for controlling stimulation impulses during a stimulation at a user 2 comprises at least one sensor 3, one data processing unit 4 and one impulse unit 5. In the embodiment shown in FIG. 1 the electrodes 8 and the sensors 3 are connected with a textile, here a sweat suit 10, and they are fixed in a lower area of the leg of the sweat suit 10. So a portable system 1 is provided which allows for the user to conduct the stimulation application without any restrictions with respect to the location and/or his or her freedom of movement. Here the sensor 3 is, for example, suitable for measuring a measuring value, in particularly the EMG activity of the user 2. Advantageously, this allows to measure an EMG activity of the user 2 and to trigger a stimulation impulse, in particular an EMS impulse, which is changed dependently on the measuring value or the control signal in one or more stimulation impulse parameters. Advantageously, in the system 1 one or more sensors 3 of the same or different type(s) can be arranged.

The data processing unit 4 is configured to compare the measuring value with a threshold value and to generate a control signal for the impulse unit 5, when the measuring value and the threshold value are in a predefinable ratio to each other. In the presently shown embodiment the impulse unit 5 and the data processing unit 4 are accommodated in a common housing which can be carried in one hand by the user 2 or, alternatively, it can be put into a bag or it can detachably be linked with the sweat suit 10. Here, the impulse unit 5 is suitable for triggering stimulation impulses and it is configured to change one or more stimulation impulse parameters dependently on the control signal.

A method in which an impulse unit triggers one or more stimulation impulses comprises at least the following steps: a) measuring of a measuring value, b) comparing the measuring value with a threshold value or determining a ratio of a desired adjustment or generation of an EMS signal, c) generating a control signal, when the measuring value and the threshold value are in a predefinable ratio to each other, and d) changing a stimulation impulse parameter dependently on the control signal.

It is also possible to define a ratio for a desired adjustment from the deviation of a measured actual value with a target value, and the EMS signal is determined dependently on this ratio.

In this case, the measuring value being measured by means of a sensor is compared with a threshold value by means of suitable algorithms. Such an algorithm may advantageously be predefined in the data processing unit or it can be adjusted and/or entered. When it is realized that the measuring value and the threshold value are in a predefined ratio to each other, then a respective control signal is generated and an impulse parameter is changed dependently on said control signal. A corresponding stimulation impulse with changed impulse parameter can then be triggered by the impulse unit. Thus, for example, the intensity of the stimulation impulse can be increased or decreased dependently on the measuring value. Also, alternatively or in addition, further stimulation impulse parameters such as impulse type, intensity, duration of the stimulation impulse, frequency, slope, rest period of impulse, width of single impulse and/or duration of single impulse can be changed.

The system 1 shown in FIG. 1, in addition, comprises a user interface 6 with an input means 62, for example keys. In the embodiment shown the user interface 6 is arranged in a housing which is different from that of the data processing unit 4 and the impulse unit 5 and it is designed as a remote control device. So the data processing unit 4 and the impulse unit 5 can be controlled and adjusted by means of the remote control device comprising the user interface 6 without the necessity that the user 2 during the stimulation application has to carry the remote control device on his or her. The portable housing comprising the data processing unit 4 and the impulse unit 5 further comprises an energy source 7.

As can be seen in FIG. 2, the textile 10 may also be designed as a top. Here the electrodes 8 and the sensors 3 are arranged in a left and a right abdominal region each. Furthermore, a difference between the embodiment shown in FIG. 2 and the embodiment shown in FIG. 1 is that as a visualization unit 61 and an input means 62 a mobile phone or a tablet PC is used. In this case, the transmission of data from the visualization unit 61 and the input means 62 to the data processing unit/control unit 4 is realized by means of suitable transmission means, such as for example by radio or WLAN. An internet connection may be realized via the mobile phone. Accordingly, for example, a trainer can monitor the success of the training from virtually any arbitrary location and he or she can intervene in a corrective manner. For example, the trainer can increase the training challenges step by step.

Preferably, the control unit 4 comprises an assembly for the generation of the electric signal of the electrostimulation. When a plurality of electrodes, such as for example at least three electrodes, or a plurality of pairs of electrodes is connected with the control unit 4, then preferably a switch in the control unit may be provided which can connect the different electrodes with the assembly for the generation of the electrostimulation in a temporal offset.

In the embodiment shown in FIG. 3 as visualization unit 6 a screen 61 is provided which inter alia comprises a camera 62 as an input means 62. As can be seen directly in FIG. 3, for the user 2 a virtual reality is provided by means of the screen 61 which shows the user 2 during performing a sequence of movement, here the lifting of a weight. In this case, in the virtual environment to the picture of the user 2 taken by the camera 61 the weight is added as a part of the virtual environment. In this case for the user 2 in real-time his or her sequence of movement together with the visualized weight is shown. According to FIG. 3, here, the system 1 comprises a textile 10 in the form of a wing at which the electrodes 8 and the sensors 3 are arranged in the back area of the upper arms each. When the sequence of movement which is stored in the data processing unit 4 is not correctly performed by the user 2, then the user 2 gets a stimulation impulse via the electrodes 8. It is also possible to provide a stimulation impulse as simulation of the game situation, for example the implication of the lifted weight.

FIG. 3 shows different muscle groups. So on the one hand electrodes 8 are shown which are assigned to the muscles of the arm (e.g. for exercising the biceps). In addition, two back electrodes 20 are shown which are arranged at the back of the exercising person. The back comprises different muscle groups. On the one hand, there are the large back muscles, and under them the deep musculature can be found. The deep musculature is directly connected with the vertebrae and it is of high importance with respect to the generation of back pain. Each of these muscle groups requires specific parameters of stimulation. For example, for the biceps frequencies of lower than 100 Hz are reasonable. For the back musculature and in particularly for the deep musculature frequencies being considerably higher, such as for example higher than 1000 Hz, are necessary. The control unit 4 is configured to change during a fast switch procedure one or diverse parameters of the stimulation. For example, correspondingly the frequency can be changed. Via the already mentioned switch dependently on an electrostimulation generated specifically for a certain body region each the respective electrode(s) 8, 20 can be connected with the control unit so that this electrostimulation is transmitted to the electrodes 8, 20.

The control unit 4 is configured such that at least a learner mode and an expert mode are provided. In the learner mode conditions which for the user may result in deleterious effects cannot be adjusted. For example, the power of the electrostimulation may be limited in this case. It is also possible that the usable frequency is restricted. So, in particular, in the back part the exercise frequency should not be chosen too low.

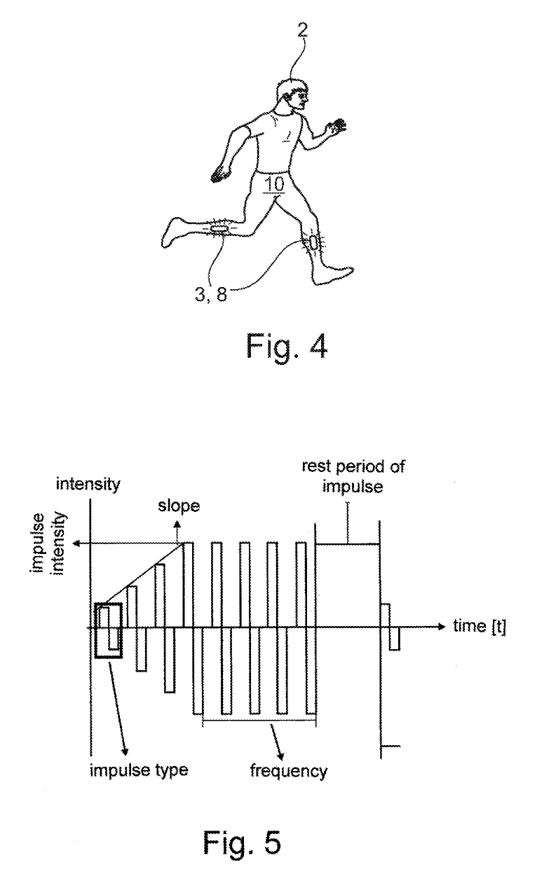

FIG. 4 shows an illustration for controlling stimulation impulses with an EMS user 2 who is equipped here with at least 2 electrodes at sweatpants 10. During his or her activity he or she is stimulated by impulses. The impulses are clocked via a sensor. Here, optionally, a time, pressure, acceleration or ultrasonic sensor, a resistance apparatus or an electromyography apparatus is used.

FIG. 5 shows an illustration of a voltage characteristic of an exemplary stimulation impulse. Such a stimulation impulse may in particularly be triggered and changed in one or more stimulation impulse parameters dependently on the control signal by the impulse unit 5. Here, in FIG. 5 it can directly be seen that in this case squarewave characteristics of the impulse intensities are used each. The whole stimulation impulse comprises one pulse unit consisting of several single impulses which are triggered in quick succession with the same or different intensity. Here, each single impulse is a singular event, wherein the momentary values of them only within a limited period of time distinctly differ from zero. The intensity of the stimulation impulse is reached after a series of sloping impulses with increasing maximum magnitudes. The slope as shown in FIG. 5 shows here a rise which is reached by the maximum magnitudes of the series of such sloping impulses with increasing maximum magnitudes. In FIG. 5 after the execution of the stimulation impulse a rest period of impulse is shown which describes the period of time between two consecutive stimulation impulses. The stimulation impulse which follows after the rest period of impulse is indicated by its first sloping impulse. The stimulation impulse shown has an impulse width of about 25 to about 200 μs.

Basically, the design of the electronics of the system according to the present invention is such that up to 12 muscle groups can be exercised. For being able to drive the electrodes of the respective muscle groups independently of each other, here according to prior art up to 12 channels would have to be provided in the electronics. For being able to design the electronics and/or the control unit of the system according to the present invention relatively cost-effective for the user, an alternative system may have only one channel and comprise one relay as well as one micro controller. Also several channels, thus devices for generating the EMS signals, may be provided which are connected or connectable with at least 4, preferably at least 8 electrodes each. With them the single electrodes can be driven one after the other. Alternatively or in addition, the electrodes can arbitrarily be assigned, for example at first left abdomen—right abdomen, then left abdomen—right chest.

In particular, the system may allow at least one change of channel. A corresponding system may comprise a step of the change of channel between two or more electrodes or pairs of electrodes. Such a change of channel allows a stimulation of the whole body of the user by means of only few, preferably one sole channel electronics. A system according to the present invention preferably comprises a single channel system. In addition, a person skilled in the art will appreciate that the brain of the user, in particularly of a human user, cannot process signals in the range of milliseconds and microseconds. When in the case of such a single channel system switching between the single electrodes is performed quickly enough, such as for example each millisecond, then at a frequency of 100 Hz 10 channels can be used without any problem with only one stimulation channel, and the user will get the impression that the stimulation influences the whole body. For example, such a switching may be conducted between single electrodes or pairs of electrodes, i.e. between, for example, electrodes which are arranged at the chest of a user and, for example, electrodes which are arranged at the abdomen or between electrodes which are arranged at the right side of the chest and electrodes which are arranged at the left side of the chest.

Such a system may also comprise electrodes which are arranged at the spinal column, and it may be possible that it can switch from the upper region of the spinal column to the lower region of the spinal column, or vice versa, for example for treating back pain. Therefore, such a single channel system advantageously allows the replacement of a multi-channel system.

A change of channel may also be conducted with more than one channel electronics. This should mean that at least one channel drives at least two electrodes. A person skilled in the art will directly appreciate that advantageously the number of electrodes can be increased and (as mentioned above) the number of groups. In such a case partly a concrete assignment, but partly also a flexible one may be realized.

Therefore, particularly advantageously, a change of channel can be used to drive the impulse unit, in particularly different electrodes, with stimulation impulses. This makes it possible to trigger the stimulation impulses at impulse units, in particular electrodes, in different regions of the body of the user and thus to supply each arbitrary muscle with a stimulation impulse.

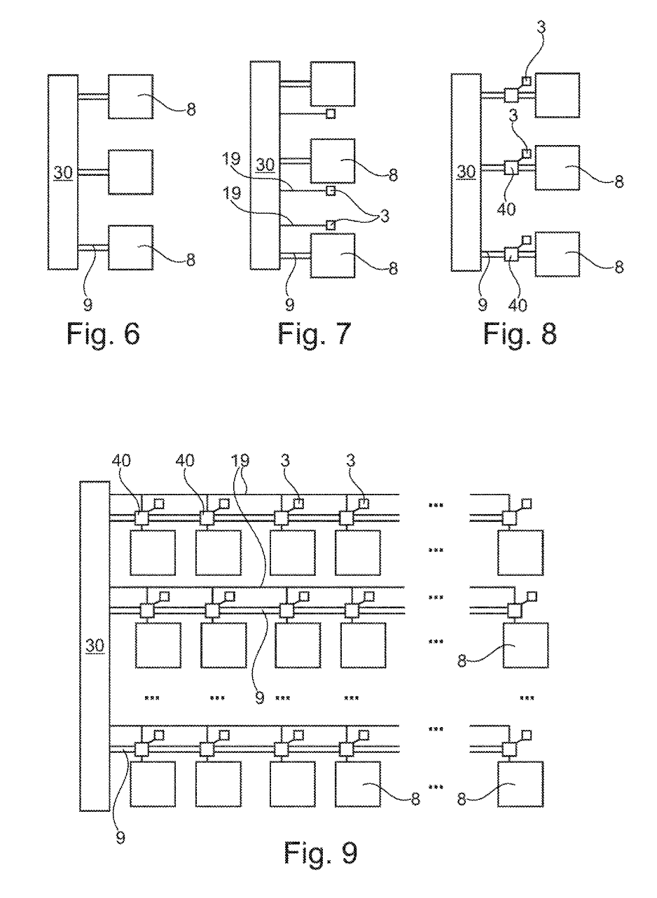

In FIGS. 6 to 9 different arrangements of the electrodes 8 with respect to the control unit 4 are shown. Here, schematically the electrodes 8 of the electrostimulation are shown and this illustration comprises different variations: On the one hand, each of the shown squares 8 may represent one pair of electrodes which are aligned in close or mediate vicinity to each other. In an alternative, each one of these squares 8 may represent one single electrode consisting of one piece. In this case a further return electrode which may also be referred to as ground electrode may be provided (for reasons of clarity, this electrode is not shown in the figures). In this second case the current flows via the respectively activated electrode 8 to said ground electrode. In an alternative or in addition, the current may also flow from one of the electrodes 8 to one of the other electrodes 8. In this case it is not necessary to provide a return electrode.

In FIG. 6-FIG. 8 three electrodes (and/or pairs of electrodes) 8 are shown each. They are exemplary for a considerably higher number of electrodes. For example, the control unit 4 may comprise a power unit for generating a stimulation impulse and a plurality of switches, such as e.g. relays, which distribute the stimulation impulses to the single electrodes. Since the stimulation impulses or durations for each single electrode are relatively short, the time between two impulses of the same electrode can be used for supplying several other electrodes with their stimulation impulses.

In FIG. 7 a variant of the embodiment of FIG. 6 is shown. Here, to each electrode 8 a sensor 3 is assigned. For signal transmission the sensor 3 is connected with the control unit 4 via a control line 19 each. In a preferable embodiment the sensors 3 may be resistance sensors which e.g. due to the conductivity recognize, whether there is a good contact between the corresponding electrode 8 and the skin. When there is no good contact, then the stimulation impulse may be amplified. In a preferable variant of this embodiment the corresponding electrode 8 itself may also fulfil the function of the sensor. In this case, as already mentioned above, the electrode 8 may consist of two parts and may be used in different operating modes. During the operation of the electrostimulation the stimulation impulse is applied via the electrode(s). In an alternative operating mode the electrode is used as a sensor. So, for example, the electric contact to the skin is measured.

In FIG. 8 a further alternative of this embodiment is shown. In this case, to each electrode 8 respectively one switch assembly 40 is assigned. In an alternative, a corresponding switch assembly may also feature a plurality of assigned electrodes. As a design feature should be mentioned that the switch assemblies 40 are formed as units which are detached and locally separated from the control unit 4. Preferably, the switch assemblies do not comprise user input or output interfaces. In an alternative embodiment, however, the switch assemblies may be provided with a lamp for showing the user, when the respective switch assembly is active. Furthermore, preferably, no input and/or output means are provided.

In alternative embodiments the switch assemblies 40 may have different designs. So, in a first variant, the switch assemblies 40 comprise an electric (electronic) switch which can be opened and closed. In the open position the stimulation impulses which are generated by the control unit 4 are transmitted to the corresponding electrode 8 via the power transmission line 9. Via the lines of the signal transmission 19 the switch assemblies 40 receive the command to open or to close the switch. One advantage of these local switch assemblies which are preferably arranged near (close proximity) the electrodes is a considerably reduced effort for wirings. Thus, for each switch assembly no special and/or separated line to the control unit 4 is necessary. In contrast to the embodiment shown in FIG. 8 one line 9 for power transmission starting from the control unit or an energy supply is enough. There is a connection of the energy supply near the electrodes between the single switch assemblies 40. The switch assemblies 40 may also be connected with one or more sensors 3 and the switching of the switch impulses onto the electrodes 8 is performed dependently on the measuring values which are received from the sensors 3.

In a second variant the switch assemblies 40 comprise a certain “switch intelligence” and/or they are configured to generate a stimulation impulse by themselves. In this case via the signal and/or control lines 19 no direct stimulation impulse is transmitted from the control unit 4. Instead of that a logic signal for activating the switch assembly 40 is transmitted. Dependent on the measuring results of the sensors 3 and on parameters of the activation which they receive from the control unit 4, the switch assemblies 40 themselves generate the power signal of the electrostimulation. For that the switch assemblies 40 must be supplied with energy. This is realized via the power transmission lines 9. For several switch assemblies one common power transmission line may be provided.

In FIG. 9 a variant is shown in which a very high number of electrodes is wired up in a matrix-like manner. Here, the number of lines 9 of the power transmission starting from the control unit 4 is considerably lower than the number of electrodes 8. In temporal succession the control unit 4 generates on each of the shown lines an electrostimulation for one or more of the connected electrodes 9. Via the control lines 19 one signal each is transmitted which determines which one of the power switches of the switch assemblies 40 should be switched on, so as to direct the electrostimulation from the line 9 to the desired electrode 8. The control line 19 may be designed as a bus which, for example, transmits data in a serial manner. In a corresponding switch logic circuit it is encoded for which one of the switch assemblies 40 the respective data package with the control commands contained therein is intended. As already explained for FIG. 8, also here in FIG. 9 the switch assemblies may be configured to generate the signal and/or the electrostimulation by themselves.

The switch assemblies may have different designs. In one variant they are optimized for having the absolutely smallest size. In this case, virtually, they only consist of the switch component which may be a transistor or another electronic switch. Thus, the volume of the switch assembly may be smaller than 0.5 cm³. In the case of a flat design they can be integrated into the clothing without any unpleasant sensation of a large “knot” for the user. In this variant, preferably, to each electrode (or each pair of electrodes) one switch assembly is assigned.

In a second variant the switch assembly may be considerably larger. In it the “switch intelligence” for a plurality of electrodes may be provided. So in the clothing several switch components may be contained. In this case the volume may be larger than 1 cm³ and smaller than 20 cm³, preferably smaller than 10 cm³. Thus, this variant of the switch assembly is so large that it can clearly be felt by the user. It is integrated in the clothing at locations which do not result in an unpleasant sensation for the user. This may be, for example, in the neck, on the chest, in the area of the belt, at wrists or ankles or, for example, at the calves. In this case, for example, at an item of clothing at least three switch assemblies may be integrated.

In both above-mentioned variants the switch assemblies preferably comprise no haptic input devices, such as switches or keys for switching on or switching off or controlling. But also a wireless signal transmission may be chosen, such as e.g. via radio (e.g. Bluetooth). So a mobile control unit may control the single switch assemblies via an intelligent control unit (e.g. smartphone).

The measuring of time is very important, since dependently on the time special controlling and adjusting possibilities are given. So within a training event a time-related adjustment is possible. So a temporal increase of the challenges can be adjusted. For example, the challenges and/or the EMS impulses can be increased by 10% every 10 minutes. Also the training challenges can be adjusted in a weekly rhythm by 5% increase per week for taking the general training success into account.

Features which are described in connection with single embodiments, particularly in connection with the embodiments of FIGS. 6 to 9, are allowed to be combined with one other without any limitations, as long as technical requirements are not necessarily an obstacle for that.

EMS apparatuses which are conventionally available on the market transmit an identical stimulation to all connected electrodes in a parallel mode. At best, only the intensity/voltage can be adjusted. This means that, for example, the musculature of the arm is stimulated with the same parameters: frequency, impulse increase, rest period of impulse, duration of impulse, impulse type, etc., as the musculature of the trunk. This is disadvantageous, because the properties of the musculature of both mentioned parts of the body are fundamentally different. While the trunk mainly is responsible for the permanent maintenance of the stability and the force transmission of the body, the arms for the main part have to execute short-time and (relatively to the mass of the muscle) very strong work. This, inter alia, is also confirmed by a comparison of the composition of the muscle fibers. While in the area of the trunk predominantly the type 1 fibers which are resistant to fatigue are present, in the musculature of the arm a relatively high proportion of quickly contracting type 2 fibers can be found. In the case of prior art EMS systems this means that the quickly powerful arms e.g. are exposed to an endurance stimulus and the trunk being resistant against fatigue is exposed to a stimulus for increasing the quickly fatiguing type 2 fibers. Thus, the stimulations used in this manner are in contrast to the function and the natural adjustment of the corresponding part of the body. For this reason the following three zones were defined for which individually adjustable parameters are possible each. The most important parameter is the frequency.

Accordingly, for the type of sport “jogging” for the following body regions the following frequency ranges were defined:

trunk: 30-50 Hz, submax. continuous impulse

neck, chest & arms: 80-100 Hz, submax. continuous impulse

legs & buttocks: 30-50 Hz, submax. continuous impulse

For other types of sport other frequency ranges were defined as being advantageous. For example, for track and field athletics (throw) the following values are valid:

trunk: 80-120 Hz, submax.-max. 5 on-20 off, rise & fall: 0.1 s

neck, chest & arms: 80-120 Hz, submax.-max. 5 on-20 off, rise & fall: 0.1 s

legs & buttocks: 80-120 Hz, submax.-max. 5 on-20 off, rise & fall: 0.1 s

Accordingly, the EMS system preferably comprises a database in which for a plurality of types of sport for at least three body regions, namely: 1: trunk, 2: neck, chest & arms, and 3: legs & buttocks, the preferable parameter ranges are defined. Dependent on personal data, such as e.g. age, gender, fitness condition, the stimulation parameters for the regions will preferably be determined in an individual manner.

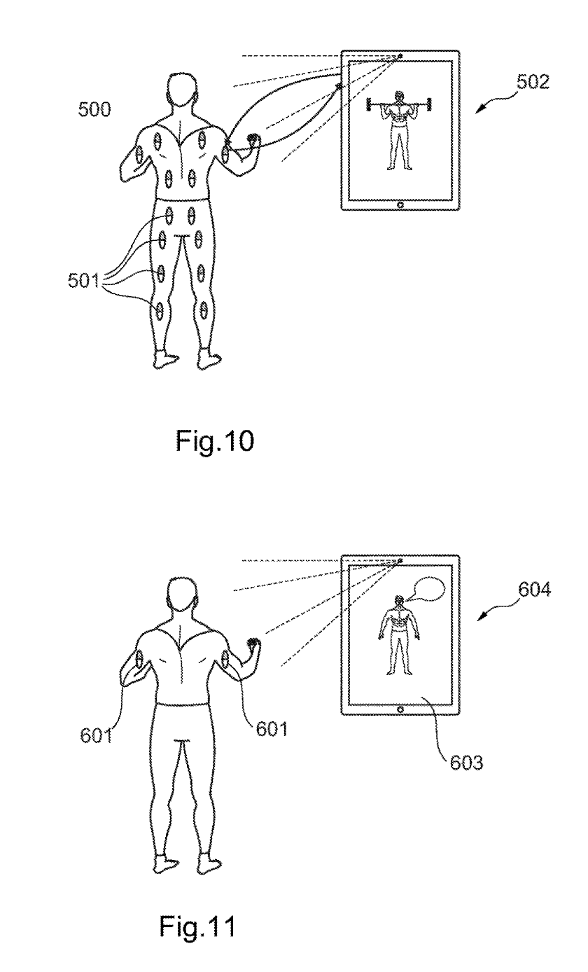

The principle sketch shown in FIG. 10 shows a therapy or training method according to the present invention. In 500 a suit with sensors 501 can be seen which can receive and/or send signals (symbolized by arrows). In the case of tensions and/or increased muscle activity the sensors can measure the activity and may analyze it with an analyzing software. When the analysis shows that a muscle is too active, then the muscle is activated on the contralateral side for initiating an inhibition, so that the muscle loses its tonus and/or becomes relaxed. The method works according to the principle of the afferent collateral inhibition. In the following, the principle of the afferent collateral inhibition is described: work of muscles (muscle contraction) is only possible, when in the case of an activation of the agonist a concurrent inactivation of the antagonist takes place, and vice versa. This is achieved by the connectivity of afferents and efferents in the spinal cord via inhibitory interneurons. In FIG. 10 the reception of the sensor data is shown, wherein the activity signals of the musculature are received and they are transmitted to a control unit, such as e.g. a mobile terminal device (smartphone, tablet PC). On the mobile terminal device 502 a software analyzing process is going on. The data are transmitted in a mobile and/or wire-connected manner. In 501 the sensors can be seen which acquire the muscle activity and send it to the mobile terminal device. The procedure of transmitting the measured data is not necessarily directly performed by the sensors, but the sensors may be connected with a data transmission unit which performs the transmission. In 501 the sensors/electrodes which transmit the muscle-stimulating stimuli onto the skin are shown. They are transmitted by the mobile terminal device 502 and/or wire-connected. In 502 the software is shown as a trainer method.

The procedure of transmitting the measured data is not directly performed by the sensors, but the sensors may be connected with a data transmission unit which performs the transmission. In this sense the single sensors are connected with a transmittance module, for example, via a data cable in the form of a data bus. The sensors/electrodes may be separated components. Preferably, the sensors may be arranged near the electrodes. In section 5d the sensors/electrodes can be seen which are activated and transmit the muscle-stimulating stimuli onto the skin. They are transmitted by the mobile terminal device 5e and/or the control unit 5e and/or wire-connected. In the case of this transmission electrodes are singly or in groups connected with a (radio) receiving/transmitting unit which receives the activating signals and activates the EMS electrodes. In 5f the software is schematically shown as a trainer method (the trainer method represents the analyzing software). It recognizes, when a modulation is required. 5b and 5e are one apparatus which is only sketchily shown in the control circuit.

The principle sketch shown in FIG. 11 shows a user with a system according to the present invention in the form of an item of clothing which is worn at the upper body and a visualization unit in the form of a screen 604 (e.g. also glasses, in particularly 3D glasses may be possible). The user interacts with the virtual world (environment). Via the visualization unit 604 a virtual trainer 603 can be seen which demonstrates an exercise and gives instructions. The exercising person repeats this exercise. The trainer gives a training instruction which should be simulated by the user. When he or she does not correctly carry out this exercise, then this is acquired via a sensor, and the software processes the signal and transmits a haptic signal (electrotactile, vibro-tactile or mechano-tactile) to the user. This signal may be an EMS signal which is configured to directly effect a muscle activation. In an alternative, a signal with a frequency which is not suitable for the muscle activation can be provided. This signal will sensitively be recognized by the body and the user can subsequently intentionally perform a corrected movement. In 603 an extract of the visualization unit 603 can be seen, wherein the user is instructed to perform the movement correctly, while the system regulates the performance of the movement via the sensors 601. The system recognizes via the sensors 601 (e.g. strain gauge strips) in the textile, whether the movement has correctly been performed. When the movement has not correctly been performed, then an avatar correctly shows the exercise in real-time. So a realistic understanding of the exercise becomes possible. Via this virtual feedback method (by means of glasses or a helmet, visor, contact lens, display being located before the eyes) each conceivable movement can be learned and also a new interaction becomes possible. In FIG. 11 an item of clothing can be seen in which single or several sensors 601 are manufactured which can send and/or receive signals. The transmitting of measured values may be achieved via a transmitting module (e.g. radio, Bluetooth) being connected with the sensor. With it also the receiving of data is possible, such as for example activation information for the single electrodes. Also vital parameters can be acquired, as described above. Also EMS signals can be transmitted from the virtual trainer 603. For the measurement of movement technically several possibilities are available (e.g. acceleration sensor, sports biomechanics). Often, miniaturized piezo-electric acceleration sensors being manufactured from silicon are used which transform the pressure fluctuations being generated by an acceleration into electric signals. Small, robust sensors are characterized by low weight of only few grams, a high sensitivity and a good resolution of the signal. Recent piezoresistive and piezo-capacitive sensors provide a signal which does not only show the acceleration, but also the inclination of the sensor (position with respect to gravitation). In horizontal or vertical position the proportions of direct current voltage (DC) of the signal are different, so that also the position of the body in the space can be determined. Gyrosensors are also capable of measuring the angular acceleration. An acceleration sensor only reacts in one dimension with maximum sensibility, so that two or three sensors have to be combined for being able to acquire movements in the plane or in the three-dimensional space. For many purposes measurements in one or two dimensions (axis) are enough, while the human movement behavior has to be measured in the three spatial dimensions (planes). The attached sketch is only for illustration, it shows only one single variant of a plurality of possible embodiment variants.

In one embodiment example a sensor, in particular a strain gauge strip, may be configured to identify the posture, such as in particular the angular position of a joint, of a person exercising with the system or to identify a movement of a part of the body or of the whole body of the exercising person and to effect an electrostimulation dependently on the posture, in particular the angular position, or the movement, in particular its velocity.

In a preferable method the point is that a training course is selected in a virtual gym. A suit, such as described above, which makes it possible to receive haptic signals is according to the present invention. Preferably, by means of a visualization unit for the user the possibility is offered to select a virtual course. The selection method may function via a gesture of the user or via a targeted movement to the respective course. The gestures are recognized via the item of clothing, particularly the suit, and are transmitted to the control unit. The control unit activates the desired function and/or the desired program. The system may comprise a user interface with a sensor which may particularly be a camera, an ultrasonic sensor or a radar sensor, and/or the user interface may be adapted for controlling the EMS system and/or single impulse parameters by gestures. For example, the visualization unit may show a direction for the user. It is possible to navigate the user and to motivate him or her to jump to the right side, the left side, ahead, back or upwards. He or she gets instructions from the virtual trainer to move. The system may also be used for learning or for online schoolings.

When a user makes a movement which is not correctly performed, then the virtual trainer recognizes that and it shows him or her the correct exercise and it gives instructions for optimizing his or her movements. The virtual trainer also simulates the movements and gives instructions for optimizing the performance of the movements. Thus, the trainer is also able to teach him or her an exercise which is specific for a type of sport, such as for example the golf swing and all conceivable movement variants. It is also possible to perform a special online-based EMS training with a virtual trainer. It is also according to the present invention to provide a mirror picture on the visualization unit for the user so that he or she can orientate visually. The method recognizes the performance of the movement, compares it with the help of the software and makes correction instructions via the virtual trainer.

### LIST OF REFERENCE SIGNS

1 system
2 user
3 sensor
4 data processing unit, control unit
5 impulse unit
6 user interface
61 visualization unit
62 input means
7 energy source
8 electrode
9 line (power transmission)
10 textile
20 back electrode
19 control line (signal transmission)
40 switch assembly
61 visualization unit, screen
62 input means, camera

What is claimed is:

1. A system for controlling stimulation impulses, comprising: at least one control unit and one item of clothing having a plurality of electrodes for electrostimulation, wherein the control unit is configured to carry out electrostimulations with defined parameters at different electrodes and during a training session different parameters can be produced at different electrodes by said control unit, wherein the item of clothing comprises data lines for transmitting measuring values and/or control signals and power lines for transmitting power for stimulation impulses, wherein the power lines have a larger cross-section than the data lines.

2. The system according to claim 1, wherein the parameters are an impulse type, a frequency, an intensity, a polarity, a duration of impulse and a rest period between impulses.

3. The system according to claim 1, wherein the control unit comprises at least one generating device for generating impulses of the electrostimulation and a distributing device is configured to distribute the electrostimulation to different electrodes.

4. A system for controlling stimulation impulses during a stimulation carried out at a user, comprising: an item of clothing, at least one sensor, at least one data processing unit and at least one impulse unit, wherein
a) the at least one sensor is suitable for measuring at least one measuring value,
b) the data processing unit is configured to compare the at least one measuring value with one threshold value each and to generate control signals for the impulse unit, when the measuring value(s) and the threshold values are in a predefinable ratio to each other,
c) the impulse unit is suitable for triggering stimulation impulses and is configured to change one or more stimulation impulse parameters dependently on the control signal,
wherein the item of clothing comprises data lines for transmitting measuring values and/or control signals and power lines for transmitting power for stimulation impulses, wherein the power lines have a larger cross-section than the data lines.

5. The system according to claim 1, wherein the control unit comprises a first mode which in particular can be referred to as a learner mode and a second mode which in particular can be referred to as an expert or trainer mode, wherein for at least one parameter of the electrostimulation in the first mode the range of the adjustable values is smaller than in the case of the second mode.

6. The system according to claim 1, wherein the system comprises at least one sensor and the control unit is configured to change at least one parameter of the electrostimulation dependently on measuring results of said sensor.

7. The system according to claim 1, wherein at least one electrode or a plurality of electrodes is non-interchangeably assigned to one or more channels.

8. The system according to claim 1, wherein at least one generated stimulation signal can be conducted to one channel and can be switched over from said channel to a plurality of electrodes or pairs of electrodes in temporal succession, wherein particularly but not necessarily at the different electrodes or pairs of electrodes different parameters of the stimulation are possible.

9. (canceled)

10. The system according to claim 1, comprising an item of clothing, wherein the item of clothing comprises switch assemblies at at least two locations near one or more respective electrode(s), wherein each switch assembly comprises at least one power switch element, such as in particular a relay, a transistor or the like, and the switch assembly is configured to actuate the power switch element dependently on measuring data provided by a sensor and/or dependently on control information provided by the control unit so as to supply the respective electrode(s) with an electrostimulation, wherein in particular at least one sensor is assigned to said switch assembly.

11. The system according to claim 10, wherein the switch assembly is smaller than 2 cm³ and in a further preferable embodiment smaller than 0.6 cm³.

12. The system according to claim 1, wherein the item of clothing comprises an electrode array of single electrodes, wherein the electrode array in particular comprises at least eight electrodes and the system is configured to provide during a training session stimulation impulses for each of these electrodes, comprising parameters which are different in groups or entirely individually.

13. The system according to claim 1, wherein in the data processing unit a ratio for the adjustment of at least two stimulation impulse parameters is specified and the adjustment of these parameters according to this ratio is carried out, when measuring values of one or more sensors are changed, wherein the stimulation impulse parameters may be parameters for the same or different electrodes.

14. The system according to claim 1, wherein one or more sensor(s) are configured to receive different measuring values, wherein in particular the measuring principle of these sensors is based on different physical principles and the data processing unit is configured to evaluate these measuring values by the help of comparison and to trigger stimulation impulses from this and, in doing so, to change stimulation impulse parameters.

15. The system according to claim 1, wherein one or more sensor(s) are configured to diagnose tensions in a muscular tissue, in particular via the measuring principle of a bioelectric impedance analysis (BIA), a sensor for oxygen saturation, an electromyography sensor and/or a calorie consumption sensor, and that the control unit is configured to define muscles dependently on these measuring results which have to be activated for reducing these tensions and that the control unit is further configured to send respective commands of the electrical muscle stimulation to the electrodes which are assigned to the muscles to be activated.

16. (canceled)

17. The system according to claim 1, wherein the system is configured to generate a plurality of stimulation impulses in temporal succession, wherein at least one parameter, in particular the intensity, of each impulse is different from the directly preceding impulse so that undulating characteristics of the impulse parameters, in particular also superimposed waves can be adjusted.
